(19) United States
(12) Patent Application Publication
(10) Pub. No.: US 2018/0289506 A1
(43) Pub. Date: Oct. 11, 2018

(54) CAGE INSERTED BETWEEN VERTEBRAE

(71) Applicant: SOLCO BIOMEDICAL CO., LTD., Pyeongtaek-si (KR)

(72) Inventors: Seo Kon Kim, Anyang-si (KR); Il Kim, Seoul (KR); Myung Heon Ha, Osan-si (KR)

(21) Appl. No.: 15/544,463

(22) PCT Filed: Feb. 23, 2017
(86) PCT No.: PCT/KR2017/001984

§ 371 Date: Jun. 25, 2018

(30) Foreign Application Priority Data
Jan. 31, 2017 (KR) 10-2017-0014056

(51) Int. Cl. A61F 2/44

(52) U.S. Cl. CPC A61F 2/4465; A61F 2002/30843; A61F 2310/00023

(57) ABSTRACT

The inventive concept relates to a cage inserted between vertebrae, and more particularly, to a cage inserted between vertebrae, which implements an articulated rotary motion and may be stably inserted between vertebrae. A cage inserted between vertebrae according to the inventive concept includes a cage body inserted between vertebrae, and a rotary member connected to the cage body to be rotatable and fastened to an inner rod of a cage inserting apparatus. The rotary member includes a connector connected to the cage body, and a screw thread extending from an end of the connector to protrude toward an outside of the cage body and screw-fastened to the inner rod.

### BACKGROUND

The inventive concept relates to a cage inserted between vertebrae, and more particularly, to a cage inserted between vertebrae, which may implement an articulated rotary motion and may be stably inserted between vertebrae.

An intervertebral disc is a circular cartilage disc inserted between vertebrae, and performs a shock absorbing function of absorbing a weight of and a shock on a body at a location between vertebrae except for some of cervical vertebrae and dispersing the shock, which is like a spring. In this case, the intervertebral disc functions to hold the vertebrae such that the vertebrae are not dislocated, make the range of an intervertebral foramen smooth by separating two vertebrae such that a vertebral nerve is not compressed, and make movement of each vertebra smooth.

Such an intervertebral disc includes an annulus fibrosus and a nucleus pulposus. The annulus fibrosus adjusts movement of pieces of the vertebrae, and the nucleus pulposus therein has 70 to 80% of water content. The intervertebral disc absorbs or transfers a weight and a shock that are vertically applied. In degenerative disc diseases, movement of the annulus fibrosus or capability for the annulus fibrosus to contain the nucleus pulposus is weakened, and the water content of the annulus fibrosus is reduced. Due to such a complex result, diseases such as spinal stenosis, forming of osteophyte, disc prolapse and compression of a nerve root occur.

An example of a method for treating such diseases accompanied by the intervertebral disc includes a method for replacing a space between two adjacent vertebral bodies with an artificial disc or an implant, for example, a so-called cage after a damaged intervertebral disc of a human body is removed. That is, as the cage is implanted, a natural state is made as much as possible. An original distance between the two adjacent vertebral bodies, which is an original height of the intervertebral disc, is restored, so that a function of the vertebrae is restored.

In recent years, transforaminal lumbar interbody fusion (TLIF) is proposed as a surgery method for inserting such a cage between vertebrae. The TLIF, which is one of vertebral body fusions, is a surgery method for approaching a rear side of the human body and inserting a cage. In detail, the TLIF is a surgery of inserting a cage using an insertion device while removing vertebral joints in a direction in which a neuropore protrudes after a surgical site is incised along opposite sides of a spinal muscle and is minimally exposed such that a screw may be fixed. The TLIF has advantages in that an amount of bleeding is small and an operation time may be shortened.

In the TLIF, for minimal incision and minimization of interference in the human body, generally, after a tip end of the cage is firstly inserted through a rear surface (a side of a back) of the human body and is located between the vertebrae, a side surface of the cage is arranged toward a front surface (a side of an abdomen of the human body) of a portion between the vertebrae, and thus, the insertion of the cage is completed. That is, an impactor which is an auxiliary tool is required such that the side surface of the inserted cage faces the front surface of the portion between the vertebrae. Force is applied to the side surface of the cage using the impactor to rotate the cage, so that the cage may be arranged. However, a surgery using the impactor has a disadvantage in that the surgery is difficult or success in the surgery cannot help depending on an operational ability of an operator.

Technologies disclosed in Korean Patent No. 10-1273199 (hereinafter, referred to as “Patent document 1”) and Korean Patent No. 10-0371308 (hereinafter, referred to as “Patent document 2”) are proposed as a method for facilitating location adjustment of the cage. An apparatus for inserting a cage according to the prior arts is configured to implement an articulated rotary motion of pivoting a cage after inserting the cage such that a tip end of the cage faces a front surface of a portion between vertebrae, which is a so-called articulating mechanism.

In the apparatus for inserting a cage in which such an articulating mechanism is implemented, it is very important that the cage keeps a standing posture without rotation while the cage is inserted. In addition, it is very important that coupling force between the cage and a cage inserting apparatus is improved even while the height (thickness) of the cage is minimized depending on requirement, so that stable insertion may be achieved. Further, various researches and developments are performed to solve the above-described technical problems.

### SUMMARY

The inventive concept is conceived to solve the above technical problems, and an aspect of the inventive concept is to provide a cage inserted between vertebrae, which may implement an articulated rotary motion, and may be stably inserted by improving coupling force between the cage and an apparatus for inserting a cage.

Technical aspects to be achieved by the inventive concept are not limited to the above-described technical aspects, and not-mentioned other technical aspects should be clearly understood by those skilled in the art to which the inventive concept pertains, based on the following description.

To implement the above-described problems, disclosed is a cage inserted between vertebrae, the cage including: a cage body inserted between vertebrae; and a rotary member connected to the cage body to be rotatable and fastened to an inner rod of a cage inserting apparatus, wherein the rotary member includes: a connector connected to the cage body; and a screw thread extending from an end of the connector to protrude toward an outside of the cage body and screw-fastened to the inner rod.

According to the cage inserted between vertebrae according to the inventive concept, the connector may be connected to the cage body through a pin member, and the rotary member and the pin member may be formed of a material having a strength that is higher than that of the cage body. For example, the cage body may be formed of polyetheretherketone (PEEK), and the rotary member and the pin member may be formed of a titanium alloy.

According to the cage inserted between vertebrae according to the inventive concept, the screw thread may include a male screw thread fastened to a female screw thread formed in the inner rod.

According to the cage inserted between vertebrae according to the inventive concept, first and second pin markers having a shape of a pin may be installed in the cage body, and the pin member may be arranged to form a triangular structure together with the first and second markers to function as a marker.

According to the cage inserted between vertebrae according to the inventive concept, a slot providing a rotation space for the rotary member may be formed at a rear end of the cage body, and a first support wall supported by a push bar of the cage inserting apparatus and a second support wall supported by a support bar of the cage inserting apparatus may be formed in the slot.

According to the cage inserted between vertebrae according to the inventive concept, the first and second support walls may be formed at specific angles with respect to a coupling direction of the cage inserting apparatus, respectively. For example, the first support wall may be inclined at 45 degrees with respect to the coupling direction of the cage inserting apparatus, and the second support wall may be inclined at 90 degrees with respect to the coupling direction of the cage inserting apparatus.

According to the cage inserted between vertebrae according to the inventive concept, uneven patterns may be repeatedly formed on a surface of the cage body, and the uneven patterns may be formed to have a triangular shape when viewed from a front-rear direction along an insertion direction of the cage body, and have a trapezoidal shape when viewed from a side direction that is perpendicular to the front-rear direction.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a perspective view illustrating a cage inserted between vertebrae according to an embodiment of the inventive concept;

FIG. 2 is an exploded perspective view illustrating the cage inserted between vertebrae, which is illustrated in FIG. 1;

FIG. 3 is a plan view illustrating the cage inserted between vertebrae, which is illustrated in FIG. 1;

FIG. 4 is a sectional view illustrating the cage inserted between vertebrae, which is illustrated in FIG. 3;

FIG. 5 is a perspective view illustrating an apparatus for inserting a cage between vertebrae according to the embodiment of the inventive concept;

FIG. 6 is a sectional view illustrating an operating state of the apparatus of FIG. 5 for inserting a cage;

FIG. 7 is a view illustrating a change in a location of a first support wall according to rotation of the cage;

FIG. 8 is a view illustrating a process of inserting the cage according to an operation of the apparatus of FIG. 5 for inserting a cage;

FIG. 9 is a front view illustrating the cage inserted between vertebrae, which illustrates a shape of an uneven pattern illustrated in FIG. 1; and

FIG. 10 is a side view illustrating the cage inserted between vertebrae, and illustrates the shape of the uneven pattern illustrated in FIG. 1.

### DETAILED DESCRIPTION

Hereinafter, a cage inserted between vertebrae, which is related to the inventive concept, will be described in more detail with reference to the accompanying drawings.

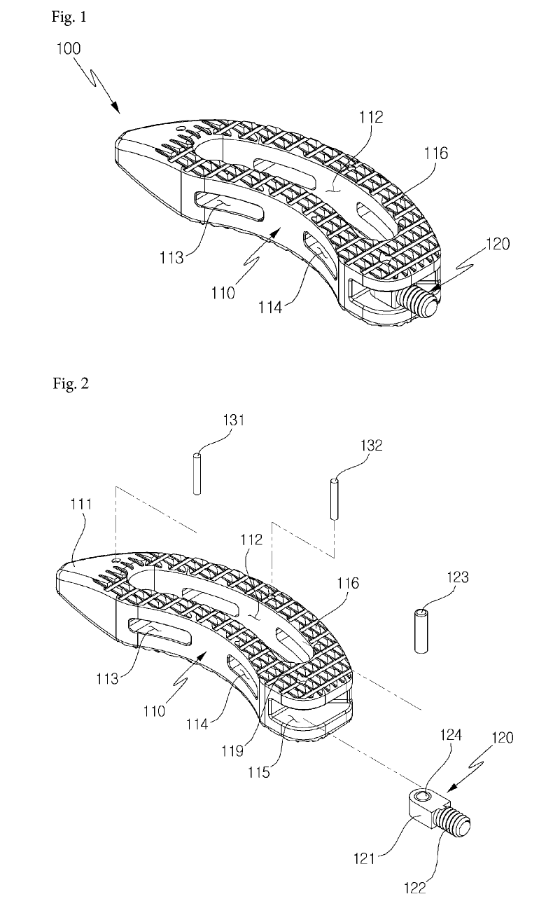

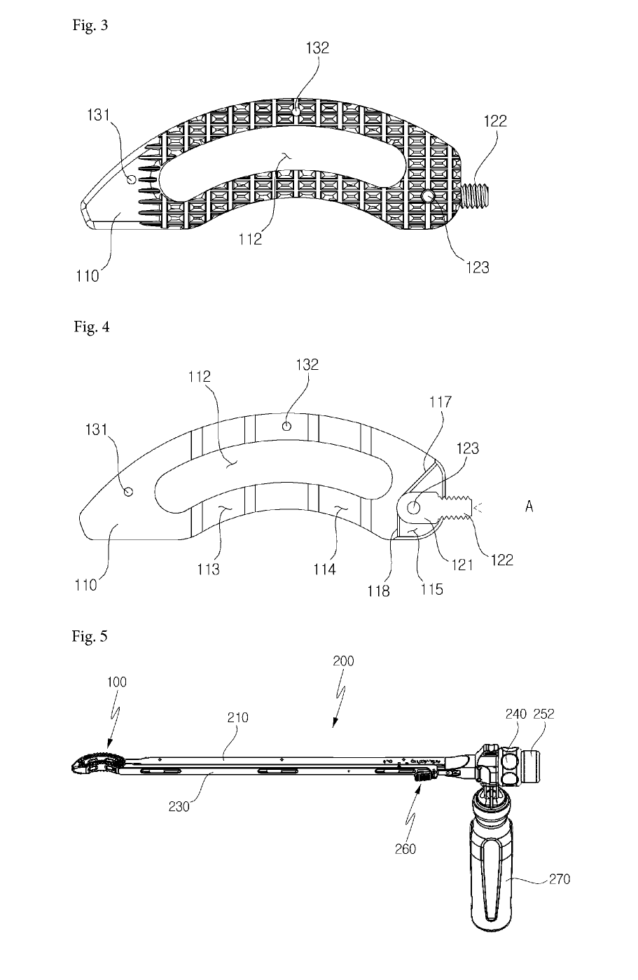

FIG. 1 is a perspective view illustrating a cage inserted between vertebrae according to an embodiment of the inventive concept, and FIG. 2 is an exploded perspective view illustrating the cage inserted between vertebrae, which is illustrated in FIG. 1. Further, FIG. 3 is a plan view illustrating the cage inserted between vertebrae, which is illustrated in FIG. 1.

Referring to FIGS. 1 to 3, a cage (100) inserted between vertebrae includes a cage body (110) and a rotary member (120) coupled to the cage body (110) to be rotatable.

The cage body (110) defines an outer appearance of the cage (100), and in detail, has a constant thickness, that is, a thickness close to a not-damaged disc, inserted between vertebrae. The cage body (110) has an anatomically rounded shape such that the cage body (110) is suitably inserted between vertebrae. That is, a side portion of the cage body (110), which corresponds to an abdomen of a human body after the insertion, is convex and a side portion of the cage body (110), which corresponds to a back of the human body after the insertion, is concave, and the cage body (110) may have an overall rounded shape.

A tip end of the cage body (110) may be inclined and an inclined surface (111) of the tip end may be formed to be vertically symmetric such that insertion may be easily performed. Uneven patterns (116) configured to prevent sliding at contact with a vertebral body (10) may be repeatedly formed on upper and lower surfaces of the cage body (110), and may function to make fusion with the vertebral body (10) firmer as well as to prevent the sliding. The shape of the uneven patterns (116) will be described below in detail.

A powdered bone accommodating hole (112) in which powdered bones are accommodated may be vertically formed through the cage body (110), and a plurality of side through-holes (113 and 114) allowing the powdered bones to be more smoothly accommodated may be formed on side surfaces of the cage body (110). Through such side through-holes (113 and 114), the powdered bones may be smoothly accommodated, and a gap (empty space) may be effectively prevented from being generated, by increasing the density of the accommodated powdered bones.

The rotary member (120) is connected to a rear end of the cage body (11) to be rotatable. A slot (115) defining a rotation space for the rotary member (20) is provided at the rear end of the cage body (110) and the rotary member (20) is connected to the cage body (110) to be rotatable inside the slot (15).

The rotary member (120) includes a connector (121) connected to the cage body (110) and a screw thread (122) extending from an end of the connector (121).

The connector (121) may have a rounded end such that the connector (121) is suitable for a rotating motion and may be connected to the cage body (110) by a pin member (123). An insertion hole (124) into which the pin member (123) is inserted may be formed through the connector (121), and the pin member (123) passes through a fastening hole (119) formed in the cage body (110) to be inserted through the insertion hole (124) of the connector (121) so as to form a connection structure of the connector (121).

The screw thread (122) may extend from the end of the connector (121) to protrude toward the outside of the cage body (110), and may be fastened to an inner rod (250) of a cage inserting apparatus (200) (see FIG. 5), which will be described below.

The screw thread (122) has a shape in which a screw thread is formed on a cylindrical member extending from the connector (121). According to the present embodiment, the screw thread (122) includes a male screw thread fastened to a female screw thread formed in the inner rod (250). However, the inventive concept is not limited thereto, and a female screw thread may be formed in the screw thread (122) and a male screw thread may be formed on the inner rod (250).

In this way, through a structure in which the screw thread (122) of the rotary member (120) protrudes toward the outside of the cage body (110), even when the height of the cage body (110) is minimized, the diameter (for example, a screw thread of M 3.0 or more may be used) of the screw thread (122) may be increased regardless of the thickness of the cage body (110). Accordingly, coupling force with the cage inserting apparatus, particularly, the inner rod (250) may be more improved, and the improved coupling force enables stable insertion when the cage (100) is initially inserted. Because the rotary member (120) remains between vertebrae even after surgery is completed, it is appropriate in terms of a decrease in the diameter that the male screw thread is applied to the screw thread (122).

Meanwhile, according to the present embodiment, the rotary member (120) and the pin member (123) may be formed of a material that is stronger than that of the cage body (110). For example, the cage body (110) may be formed of a resin material having bio-compatibility for a human body and having properties close to that of a bone, such as polyetheretherketone (PEEK), and the rotary member (120) and the pin member (123) may be formed of a titanium alloy (for example, Ti6Al4V ELI). Through such a configuration, the rotary member (120) and the pin member (123) may function to support a normal load that is higher than a strength of the PEEK material. In addition, the titanium alloy is used, so that additional fusion with a bone fusion material may be provided.

Meanwhile, a first marker (131) and a second marker (132) having a shape of a pin are installed in the cage body (110), and a location and a posture of the cage (100) may be easily identified using the first and second markers (131 and 132) through radiation during surgery. The first and second markers (131 and 132) may be formed of a radiation-impermeable material, for example, a tantalum material.

According to the present embodiment, the pin member (123) for connection to the rotary member (120) is also formed of a radiation-impermeable material (for example, a titanium alloy), and thus functions as a marker. The pin member (123) is arranged to form a triangular structure together with the first and second markers (131 and 132). In the present embodiment, the first marker (131) is located at a tip end of the cage body (110), the second marker (132) is located at a central side portion of the cage body (110), and the pin member (123) is located at a rear end of the cage body (110), so that a triangular structure obtained by connecting the first marker (131), the second marker (132) and the pin member (123) to each other is achieved.

According to such a structure, the location and the posture of the cage (100) may be identified according to the shape of a radiation-detected triangle. In the present embodiment, there is an advantage in that the pin member (123) is used as both a connection unit and a marker, so that the number of markers required for manufacturing a cage may be reduced.

FIG. 4 is a sectional view illustrating the cage inserted between vertebrae, which is illustrated in FIG. 3.

Referring to FIG. 4, a first support wall (117) and a second support wall (118) may be formed in the slot (115) formed at the rear end of the cage body (110). The first support wall (117) is supported by a push bar (220) of the cage inserting apparatus, which will be described below, and the second support wall (118) is supported by a support bar (200) of the cage inserting apparatus (200).

The first and second support walls (117 and 118) are formed to have specific angles with respect to a coupling direction A of the cage inserting apparatus, that is, an axial direction along an initial insertion direction of the cage. For example, the first support wall (117) is inclined at 45 degrees with respect to the coupling direction A of the cage inserting apparatus, and the second support wall (118) is inclined at 90 degrees with respect to the coupling direction of the cage inserting apparatus. Such first and second support walls (117 and 118) function to enable an articulating rotary motion of the cage (100), and to enable stable insertion when the cage (100) is initially inserted. Descriptions thereof will be made later in detail.

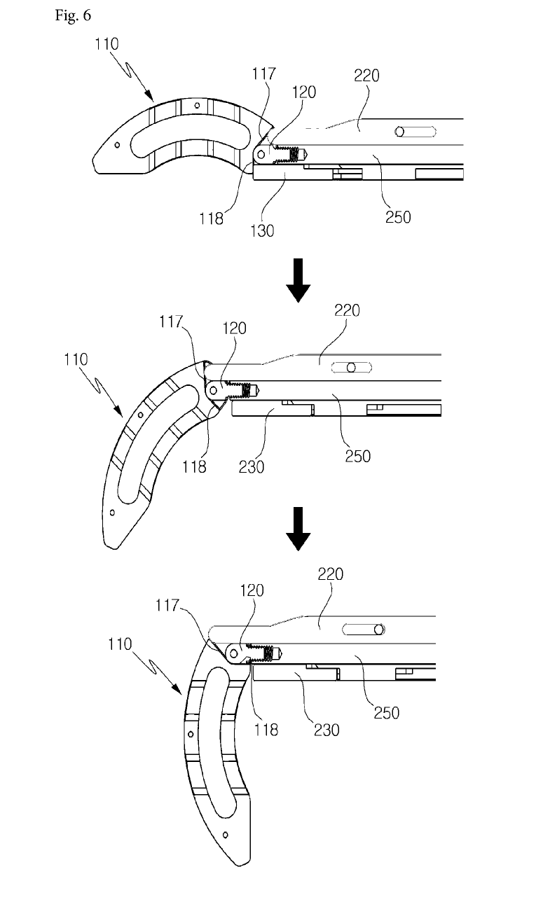

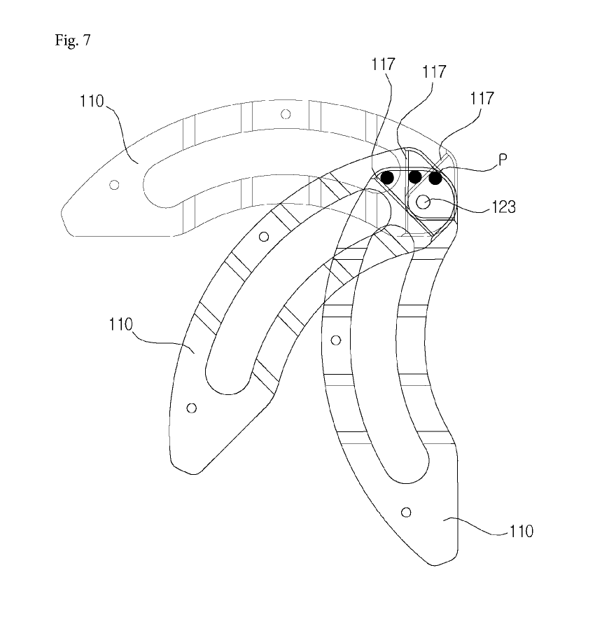

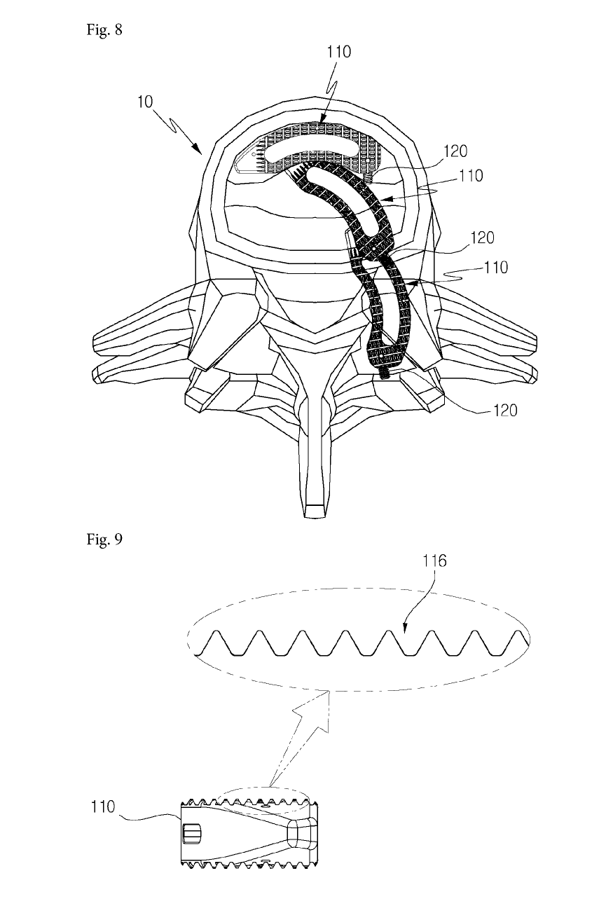

FIG. 5 is a perspective view illustrating an apparatus for inserting a cage between vertebrae according to the embodiment of the inventive concept, and FIG. 6 is a sectional view sequentially illustrating an operating state of the apparatus of FIG. 5 for inserting a cage. Further, FIG. 7 is a view illustrating a change in a location of a first support wall according to rotation of the cage, and FIG. 8 is a view illustrating a process of inserting the cage according to an operation of the apparatus of FIG. 5 for inserting a cage.

Referring to FIGS. 5 to 6, the apparatus for inserting a cage inserted between vertebrae according to the present embodiment includes a body (210), a push bar (220), a support bar (130) and an adjustment unit (140).

The body (210) accommodates the inner rod (250) coupled to the cage (100). A female screw thread fastened to the rotary member (120) of the cage (100) is formed at a tip end of the inner rod (250) and a rotation knob (252) for rotation manipulation is provided at a rear end of the inner rod (250).

The push bar (220) is installed in the body (210) to be slid. The push bar (220) is moved forwards from the body (210), that is, is moved toward the cage (100), to push the first support wall (117) of the cage (100).

The support bar (230) is installed in the body (110) in parallel to the push bar (220) to be slid, and when the push bar (220) is moved forwards, is moved rearwards in a direction that is opposite to that of the push bar (220), making it possible to implement an articulated rotary motion of the cage (100), which is a so-called articulating mechanism.

The adjustment unit (240) is installed in the body to be manipulated by a user, and adjusts movements of the push bar (220) and the support bar (230) according to manipulation by the user. In the present embodiment, the fact that the adjustment unit (240) has a rotary handle that is installed at the rear end of the body (210) to be rotatable is described as an example. Accordingly, when the adjustment unit (240) is rotated in one direction, the support bar (230) is moved rearwards and the push bar (120) is moved forwards, and when the adjustment unit (240) is rotated in a direction that is opposite to the one direction, the support bar (230) is moved forwards, and the push bar (220) is moved rearwards. Such a mechanism may be implemented by power transmission by a power transmission unit embedded in the body (210).

A locking unit (260) configured to lock and unlock a location of the support bar (230), a handle (270) that a user holds to perform surgery, and the like may be installed in the body (210).

In description of a process of operating the cage inserting apparatus, first, the user fastens the inner rod (250) and the rotary member (120) of the cage (100) to each other by rotating the rotation knob (252) of the inner rod (250), and fixes the location of the support bar (230) by operating the locking unit (160). Further, the user moves the cage inserting apparatus to an insertion position of the vertebral body (10) to insert the cage (100).

While the cage (100) is initially inserted, the push bar (220) supports the first support wall (117) formed in the slot (115) of the cage body (110), and the support bar (230) supports the second support wall (118). Because the location of the support bar (230) is fixed by the locking unit (260), the cage (101) may be stably moved by firm support force without arbitrary rotation when the cage (100) is initially inserted.

When the locking unit (260) is unlocked after the cage (100) is initially inserted, a state in which the support bar (230) may be moved is achieved. In this state, when the adjustment unit (240) is rotated, the push bar (220) is moved forwards to push the first support wall (117) of the cage body (110), and the support bar (230) is moved rearwards to provide a rotation space of the cage body (111), so that an articulated rotary motion of the cage (100) is achieved.

When the adjustment unit (140) is continuously rotated, the cage (100) is rotated up to about 90 degrees, and the second support wall (118) of the cage (100) comes into contact with a side surface of the rotary member (120), so that rotation of the cage (100) is restrained, and in this state, the cage (100) may be inserted between vertebrae.

When the cage (100) is completely inserted, the user separates the inner rod (250) from the rotary member (120) of the cage (100) by rotating the rotation knob (152) in a direction that is opposite to a direction when the rotation knob (152) is fastened, and withdraws the cage inserting apparatus from a surgical position.

FIG. 7 is a view sequentially illustrating a process of rotating the cage (100), and FIG. 8 is a view illustrating a location and a rotation state of the cage (100) corresponding thereto. In FIG. 7, a point P denotes a contact point between the push bar (220) and the first support wall (117) according to movement of the push bar (220). In this way, the push bar (220) transfers force while moving along the first support wall (117) that is inclined, so that the cage body (110) is rotated about the pin member (123). In other words, the inclined first support wall (117) functions to convert forward movement of the push bar (220) into rotational movement of the cage body (110).

The second support wall (118) is supported by the support bar (130) when the cage (100) is initially inserted, so that the cage body (110) may be stably inserted without arbitrary rotation, and functions to restrains rotation of the cage body (210) when the cage body (210) is rotated by about 90 degrees.

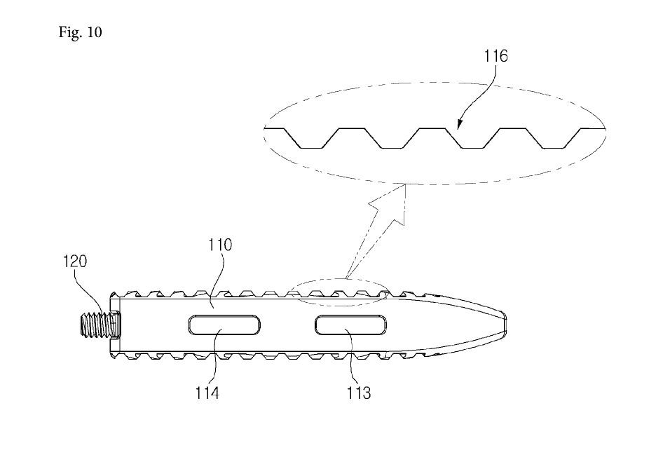

FIG. 9 is a front view illustrating the cage inserted between vertebrae, which has a shape of uneven patterns illustrated in FIG. 1, and FIG. 10 is a side view illustrating the cage inserted between vertebrae, which has the shape of uneven patterns illustrated in FIG. 1.

Referring to FIGS. 9 and 10, the repeated uneven patterns (116) may be formed on upper and lower surfaces of the cage body (110), and FIGS. 9 and 10 are enlarged views illustrating a structure of such uneven patterns (116).

According to the present embodiment, the uneven patterns (116) have a triangular shape when viewed from a front-rear direction along an insertion direction of the cage body (110), as illustrated in FIG. 9 and have a trapezoidal shape when viewed from a side direction that is perpendicular to the front-rear direction, as illustrated in FIG. 10.

According to such a structure, when the cage (100) is initially inserted, the uneven patterns (116) extend in the insertion direction, so that the uneven patterns (116) are substantially in line contact along the insertion direction. Thus, the uneven patterns (116) function as a rail, and accordingly, the cage (100) may be easily inserted.

In addition, when the cage (100) is rotated by about 90 degrees and is thus completely seated between vertebrae, the uneven patterns (116) transversely extends between the vertebrae. Thus, frictional force in a front-rear direction between the vertebrae, that is, for movement in a direction of the back and the abdomen of the human body may be increased. Accordingly, until bone fusion is completed after the cage (100) is seated, sliding of the cage (100), which may occur by lumbar flexion extension or the like, is minimized, so that the cage (100) may be prevented from being separated.

According to the inventive concept, through a structure in which a rotary member connected to a cage body to be rotatable protrudes toward the outside of the cage body, even while the height of the cage body is minimized, the diameter of a screw thread of the rotary member may be increased, so that firmer coupling force with an apparatus for inserting a cage may be provided.

Further, according to the inventive concept, first and second support walls are formed inside a slot of the cage body at predetermined angles, so that even while an articulating rotary motion of the cage is implemented, the cage may be stably inserted when the cage is initially inserted.

Further, according to the inventive concept, through uneven patterns having a shape in consideration of an insertion direction of the cage, even while the cage may be easily inserted when the cage is initially inserted, the cage may be prevented from being separated in a front-rear direction between vertebrae after the cage is seated.

The above-described cage inserted between vertebrae is not limited to the configurations and methods according to the above embodiments, but the entireties or portions of the above embodiments may be selectively combined so that various modifications may be achieved.

What is claimed is:

1. A cage inserted between vertebrae, the cage comprising:
a cage body inserted between vertebrae; and
a rotary member connected to the cage body to be rotatable and fastened to an inner rod of a cage inserting apparatus,
wherein the rotary member comprises:
a connector connected to the cage body; and
a screw thread extending from an end of the connector to protrude toward an outside of the cage body and screw-fastened to the inner rod.

2. The cage of claim 1, wherein the connector is connected to the cage body through a pin member, and
wherein the rotary member and the pin member are formed of a material having a strength that is higher than that of the cage body.

3. The cage of claim 2, wherein the cage body is formed of polyetheretherketone (PEEK), and
wherein the rotary member and the pin member are formed of a titanium alloy.

4. The cage of claim 1, wherein the screw thread includes:
a male screw thread fastened to a female screw thread formed in the inner rod.

5. The cage of claim 1, wherein first and second pin markers having a shape of a pin are installed in the cage body, and
wherein the pin member is arranged to form a triangular structure together with the first and second markers to function as a marker.

6. The cage of claim 1, wherein a slot providing a rotation space for the rotary member is formed at a rear end of the cage body, and
wherein a first support wall supported by a push bar of the cage inserting apparatus and a second support wall supported by a support bar of the cage inserting apparatus are formed in the slot.

7. The cage of claim 6, wherein the first and second support walls are formed at specific angles with respect to a coupling direction of the cage inserting apparatus, respectively.

8. The cage of claim 7, wherein the first support wall is inclined at 45 degrees with respect to the coupling direction of the cage inserting apparatus, and
wherein the second support wall is inclined at 90 degrees with respect to the coupling direction of the cage inserting apparatus.

9. The cage of claim 1, wherein uneven patterns are repeatedly formed on a surface of the cage body.

10. The cage of claim 9, wherein the uneven patterns are formed to have a triangular shape when viewed from a front-rear direction along an insertion direction of the cage body, and have a trapezoidal shape when viewed from a side direction that is perpendicular to the front-rear direction.
